(19) United States
(12) Patent Application Publication
(10) Pub. No.: US 2002/0038050 A1
(43) Pub. Date: Mar. 28, 2002

(54) Process for the manufacture of amino carboxylic acids

(76) Inventors: Bernd Siebenhaar, Kandern-Wollbach (DE); Milos Rusek, Binningen (CH)

Correspondence Address: William A. Teoli, Jr., Syngenta Crop Protection, Inc., Patent and Trademark Dept., 410 Swing Road, Greensboro, NC, 27409, US

(21) Appl. No.: 09/911,162

(22) Filed: Jul. 23, 2001

Related U.S. Application Data
(63) Continuation of application No. PCT/EP00/00434, filed on Jan. 20, 2000.

(30) Foreign Application Priority Data
Jan. 22, 1999 (CH) 127/99

(51) Int. Cl. C07C 227/02

(52) U.S. Cl. 562/526

(57) ABSTRACT

Process for the preparation of amine-group-containing carboxylic acid salts by oxidation of amine-group-containing primary alcohols in an aqueous-alkaline reaction medium, in the presence of a copper catalyst, and at an elevated temperature, which is characterized in that the copper catalyst used is a reduced copper/chrome or copper/zinc spinel.

The present invention relates to a process for the production of amine-group-containing carboxylic acid salts by oxidation of corresponding amino primary alcohols in an aqueous alkaline medium at an elevated temperature in the presence of a catalyst from the group of reduced copper spinels.

Copper-chrome and copper-zinc spinels are known commercially obtainable catalysts that are used primarily for the hydrogenation of unsaturated organic compounds, for example aldehydes or carboxylic acid esters, or for transesterification. Copper chromite was also proposed as a dehydrogenation catalyst for ethanol to acetaldehyde (see Engelhard Base Metal Catalysts; pages 1 to 24, 1991; by Engelhard, Chemical Catalyst Division, Mailand, Italy). The catalysts are usually supplied in activated form, indicating reductive treatment of the above spinels.

In J. Indian Chem. Soc., Vol. 74, pages 169-170 (1997), R. B. C. Pillai describes the disproportionisation of benzyl alcohol to benzaldehyde and toluene in the presence of a copper chromite. In this publication, the oxidation of butane-1,4-diol to succinaldehyde in the presence of copper chromite is also mentioned.

In EP-A-0 301 853, copper chromite is described as a hydrogenation catalyst, which is reduced and activated at elevated temperatures in a stream of hydrogen. During reduction, very finely divided copper is primarily separated on the surface of the catalyst particles, to which the increased activity is attributed.

The preparation of carboxylic acids from primary alcohols in the presence of activated copper spinels as oxidation catalysts has not yet been described.

It has now surprisingly been found that activated or reduced copper/chrome and copper/zinc spinels are eminently suitable as catalysts for the oxidation of amino primary alcohols to the corresponding carboxylic acids in a basic reaction medium, and that the desired carboxylic acids are formed in high yields within short reaction times because of the surprisingly high stability and selectivity of the catalyst. It was also found that the catalysts could be reused many times without significant loss of activity or selectivity, and that isolation, purification and reactivation are only indicated after several reaction cycles.

The object of the invention is a process for the preparation of amine-group-containing carboxylic acid salts by oxidation of amine-group-containing primary alcohols in an aqueous-alkaline reaction medium, in the presence of a copper catalyst, and at an elevated temperature, the process being characterised in that the copper catalyst used is a reduced copper/chrome or copper/zinc spinel.

In the context of the invention, amine-group-containing primary alcohols are also called amino primary alcohols.

The catalysts are known, commercially obtainable or obtainable by known processes, see for example EP-A-0 301 853. The reduction processes described therein may be modified in respect of temperature choice, temperature programme, choice of reduction agent and duration of reaction. Where commercial copper spinels are not activated, activation can be carried out by treating commercial copper spinel with pure hydrogen or with a mixture of a neutral gas, such as noble gases or nitrogen, and hydrogen (volume ratio for example 4:1) at a temperature of, for example, 160 to 250° C., at a constant temperature or with a temperature programme over a relatively long period, for example ca. 1 to 4 hours, and then cooling under a protecting gas (for example argon). It may be appropriate to start activation with low volumes of hydrogen, and then to increase the amounts. Afterwards, the catalyst can be used.

During activation or reduction of copper/chrome and copper/zinc spinel, the oxygen content is reduced in respect of the ideal composition of CuMe(II)O₄. The reduced copper/chrome and copper/zinc spinels to be used according to the invention may be described by the formula CuMe(II)O₄₋ₓ (formula II), in which Me is Cr or Zn and x is a number from 0.001 to 0.1, preferably 0.01 to 0.1.

The reduced copper/chrome and copper/zinc spinels may be modified in order to raise stability, selectivity or both. Suitable modifiers are, for example, divalent metals, such as manganese, nickel or in particular barium, which may be present in amounts of 1 to 15% by weight, based on the spinel. Modified copper/chrome and copper/zinc spinels are similarly commercially available.

The catalyst may be employed in a quantity of 0.1 to 40% by weight, preferably 0.5 to 30% by weight, more preferably 1 to 25% by weight, most preferably 5 to 25% by weight, based on the amino primary alcohol.

The reaction temperature may be for example from 80 to 300° C., preferably from 100 to 250° C.

The reaction is advantageously carried out under excess pressure. The pressure may be, for example, from 1 to 50 bars, preferably 2 to 25 bars, most preferably 5 to 15 bars.

The reaction is carried out in an alkaline reaction medium, preferably in the presence of NaOH or KOH. The amount of alkali base in the reaction mixture is advantageously measured such that at least equimolar amounts of alkali base are present in relation to the amino primary alcohol. It is appropriate to use an excess of alkali base, for example one to five times, preferably up to three times, most preferably up to double the molar excess.

The amino primary alcohols may contain, for example, 1 to 3 primary alcohol groups, and the amines may be primary, secondary or tertiary amines.

The amino primary alcohols may correspond, for example, to formula I,

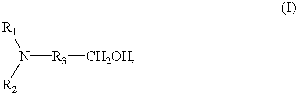

wherein R₁ and R₂, independently of one another, signify H, linear or branched C₁-C₁₈-alkyl either unsubstituted or substituted by F, Cl, Br, —NH₂, C₁-C₄-alkoxy, C₁-C₄-halogenalkyl or —COOH; C₃-C₈-cycloalkyl, C₆-C₁₀-aryl or C₇-C₁₂-aralkyl either unsubstituted or substituted by F, Cl, Br, —NH₂, C₁-C₄-alkyl, C₁-C₄-alkoxy or C₁-C₄-halogenalkyl; phosphonomethyl; R₁ and R₂ together are tetramethylene or pentamethylene; or R₁ and R₂, independently of one another, have the significance R₃—CH₂OH; and R₃ signifies linear or branched C₁-C₁₇-alkylene which is uninterrupted or is interrupted by C₃-C₈-cycloalkyl or C₆-C₁₀-aryl.

R₁ and R₂ as alkyl preferably contain 1 to 12, more preferably 1 to 8, most preferably 1 to 4 carbon atoms. Examples and preferences of alkyl have already been described.

R₁ and R₂ as cycloalkyl preferably contain 4 to 7, most preferably 5 or 6 ring carbon atoms. Examples of cycloalkyl are cyclopropyl, cyclobutyl, cyclopentyl, cyclohexyl, cycloheptyl and cyclooctyl. Cyclohexyl is preferred in particular.

R₁ and R₂ as aryl may be naphthyl and preferably phenyl.

R₁ and R₂ as aralkyl are preferably phenylalkyl. Examples are benzyl and β-phenylethyl.

R₃ as alkylene preferably contains 1 to 12, more preferably 1 to 8, most preferably 1 to 4 carbon atoms. Examples of alkylene are methylene, 1,1- or 1,2-ethylene, 1,1-, 1,2- or 1,3-propylene, 1,1-, 1,2-, 1,3- or 1,4-butylene, 1,1-, 1,2-, 1,3-, 1,4- or 1,5-pentylene, 1,1-, 1,2-, 1,3-, 1,4-, 1,5- or 1,6-hexylene, 1,1-, 1,2-, 1,3-, 1,4-, 1,5-, 1,6- or 1,7-heptylene, 1,1-, 1,2-, 1,3-, 1,4-, 1,5-, 1,6-, 1,7- or 1,8-octylene, nonylene, decylene, undecylene, dodecylene, tridecylene, tetradecylene, pentadecylene, hexadecylene and heptadecylene.

The group —R₃CH₂OH preferably signifies 4-hydroxybutyl, 3-hydroxypropyl, and most preferably 2-hydroxyethyl.

In a preferred sub-group of compounds of formula I, these correspond to formula Ia,

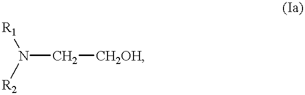

wherein R₁ and R₂, independently of one another, signify H or C₁-C₁₂-alkyl either unsubstituted or substituted by —NH₂ or —COOH; or —CH₂CH₂—OH.

In formula Ia, R₁ and R₂, independently of one another, signify H, C₁-C₄-alkyl or —CH₂—CH₂—OH. Another preferred sub-group is compounds of formula Ia, wherein R₁ signifies —CH₂CH₂—OH and R₂, independently, signifies H, C₁-C₄-alkyl or —CH₂—CH₂—OH.

Some examples of compounds of formula I are ethanolamine, diethanolamine, triethanolamine, N-methylethanolamine, N-dimethylethanolamine, N-methyldiethanolamine, N-ethylethanolamine, N-(n-propyl)ethanolamine, N-(n-propyl)ethanolamine, N-(n-butyl)ethanolamine, N-(n-pentyl)ethanolamine, N-(n-hexyl)ethanolamine, N-(n-octyl)ethanolamine, N-(n-decyl)ethanolamine, N-(n-dodecyl)ethanolamine, N-(n-tetradecyl)ethanolamine, N-(n-hexadecyl)ethanolamine, N-(n-octadecyl)ethanolamine, N-(di-n-propyl)ethanolamine, N-(di-n-butyl)ethanolamine, N-(di-n-hexyl)ethanolamine, 3-hydroxypropylamine, di-(3-hydroxypropyl)amine, tri-(3-hydroxypropyl)amine, 4-hydroxybutylamine, di-(4-hydroxybutyl)amine, tri-(4-hydroxybutyl)amine, 5-hydroxypentylamine, di-(5-hydroxypentyl)amine, tri-(5-hydroxypentyl)amine, 6-hydroxyhexylamine, di-(6-hydroxyhexyl)amine, tri-(6-hydroxyhexyl)amine, 8-hydroxyoctylamine, di-(8-hydroxyoctyl)amine, tri-(8-hydroxyoctyl)amine, 12-hydroxydodecylamine, di-(12-hydroxydodecyl)amine, tri-(12-hydroxydodecyl)amine, 18-hydroxyoctadecylamine, N-methyl-(3-hydroxypropyl)amine, N-methyl-(4-hydroxybutyl)amine, N-methyl-(6-hydroxyhexyl)amine, (2-aminoethyl)ethanolamine, di-(2-aminoethyl)ethanolamine, phosphonomethylethanolamine and diphosphonomethylethanolamine.

The compounds of formula I are known, partly commercially available or may be produced by methods analogous to those described in literature.

The process according to the invention may be carried out, for example, in such a way that the catalyst is placed in an autoclave, then first of all the primary alcohol is added, optionally in water, followed by the alkali lye, the autoclave is sealed and the reaction mixture stirred, and the reaction is commenced whilst heating. The reaction generally continues until the hydrogen generation is no longer observed. The catalyst can be decanted from the cooled reaction mixture and used for the next reaction. The alkali metal salts of the carboxylic acids thus formed may be isolated in conventional manner and purified if necessary. The salts may also be converted into the free carboxylic acids and derivatives thereof, such as acid amides and acid esters. The process according to the invention is suitable for production on an industrial scale.

The aminocarboxylic acids that may be produced according to the invention can be used for many purposes. Glycine is employed for food production. Aminocarboxylic acids are known complexing agents, which are used in the detergent industry and in water treatment. In addition, the amino alcohols may be used in the production of ionic surfactants. The amino alcohols are also valuable intermediates in the production of pharmaceutical and pesticidal compositions.

The following examples illustrate the invention more fully.

### EXAMPLE 1

### Oxidation of Diethanolamine

a) Preparation of the Catalyst

8.1 g of copper/chrome spinel catalyst with 11% by weight Ba as modifier (type G22, Süd-chemie) are reduced at 200° C. over the course of 2 hours in a stream of hydrogen (20 ml/min.). The catalyst is then transferred to a 0.3 l Hastelloy B autoclave under a protecting gas.

b) Oxidation of Diethanolamine

To the catalyst are added 42.8 g of diethanolamine (0.4 moles), 20 ml of water and 38 g of NaOH (0.95 moles) in the form of a 50% aqueous solution. Afterwards, heating is effected to 160° C. (9.5 bars, pressure resistance valve) and stirring takes place until the hydrogen generation is no longer observed (480 minutes). The yield of iminodiacetic acid or iminodiacetic acid disodium salt according to NMR analysis is 76% by weight.

### EXAMPLE 2

### Oxidation of Diethanolamine

a) Preparation of the Catalyst

8.2 g of coper/zinc spinel catalyst (type T2130, Süd-Chemie) are reduced at 200° C. over the course of 2 hours in a stream of hydrogen (20 ml/min.). The catalyst is then transferred to a 0.3 l Hastelloy B autoclave under a protecting gas.

b) Oxidation of Diethanolamine

To the catalyst are added 42.8 g of diethanolamine (0.4 moles), 20 ml of water and 38 g of NaOH (0.95 moles) in the form of a 50% aqueous solution. Afterwards, heating is effected to 160° C. (9.5 bars, pressure resistance valve) and stirring takes place until the hydrogen generation is no longer observed (380 minutes). The yield of iminodiacetic acid or iminodiacetic acid disodium salt according to NMR analysis is 97% by weight.

c) Reuse of the Catalyst

The autoclave containing the reaction mixture is cooled to 100° C. The supernatant solution is suctioned off through a riser, and the copper/zinc spinel catalyst remains in the autoclave. Then, diethanolamine and NaOH are added in the above-described proportions and reacted under the specified conditions. Up to the tenth reuse, the catalyst shows no activity loss (6^{th} reuse 330 minutes, yield 95% by weight; 10^{th} reuse 330 minutes, yield 95% by weight), and selectivity is virtually maintained.

What we claim is:

1. Process for the preparation of amine-group-containing carboxylic acid salts by oxidation of amine-group-containing primary alcohols in an aqueous-alkaline reaction medium, in the presence of a copper catalyst, and at an elevated temperature, which is characterised in that the copper catalyst used is a reduced copper/chrome or copper/zinc spinel.

2. Process according to claim 1, in which the reduced copper/chrome or copper/zinc spinel corresponds to formula II CuMe(II)O₄₋ₓ, wherein Me is Cr or Zn and x signifies a number from 0.001 to 0.1.

3. Process according to claim 2, in which x signifies a number from 0.01 to 0.1.

4. Process according to claim 1, in which the catalyst is used in an amount of 0.1 to 40% by weight based on the amino primary alcohol.

5. Process according to claim 4, in which the catalyst is used in an amount of 0.5 to 30% by weight.

6. Process according to claim 1, in which the reaction temperature is from 80 to 300° C.

7. Process according to claim 1, which is carried out at a pressure of 1 to 50 bars.

8. Process according to claim 1, in which the alkaline reaction medium is formed by adding NaOH or KOH.

9. Process according to claim 1, in which the amount of alkali base in the reaction medium is measured such that a molar excess of one to five times, based on the amino primary alcohol, is present.

10. Process according to claim 8, in which the amino primary alcohol corresponds to formula I, 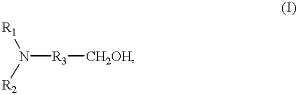
wherein R₁ and R₂, independently of one another, signify H, linear or branched C₁-C₁₈-alkyl either unsubstituted or substituted by F, Cl, Br, —NH₂, C₁-C₄-alkoxy, C₁-C₄-halogenalkyl or —COOH; C₃-C₈-cycloalkyl, C₆-C₁₀-aryl or C₇-C₁₂-aralkyl either unsubstituted or substituted by F, Cl, Br, —NH₂, C₁-C₄-alkyl, C₁-C₄-alkoxy or C₁-C₄-halogenalkyl; phosphonomethyl; R₁ and R₂ together are tetramethylene or pentamethylene; or R₁ and R₂, independently of one another, have the significance R₃—CH₂OH; and R₃ signifies linear or branched C₁-C₁₇-alkylene which is uninterrupted or is interrupted by C₃-C₈-cycloalkyl or by C₆-C₁₀-aryl.
